(19) United States
(12) Patent Application Publication
(10) Pub. No.: US 2014/0128676 A1
(43) Pub. Date: May 8, 2014

(54) Non-Rebreathing Mask With Closeable Aperture and Auxilliary Flow Tube

(71) Applicant: Barbara Law, Fayetteville, GA (US)

(72) Inventor: Barbara Law, Fayetteville, GA (US)

(21) Appl. No.: 13/669,577

(22) Filed: Nov. 6, 2012

(51) Int. Cl. A61M 16/06; A61M 15/08; A61B 1/00; A61M 16/08; A61M 16/20

(52) U.S. Cl. 600/114; 128/205.13

(57) ABSTRACT

A non-rebreathing oxygen mask for supplying a continuous flow of oxygen to a patient. The non-rebreathing oxygen mask includes a face mask portion for enclosing the mouth and nose of a patient, an oxygen reserve bag for receiving a supply of oxygen from an external source, and an intermediate oxygen entry port for regulating the flow of oxygen into the oxygen reserve bag from an external source. The mask further includes a closeable aperture through which an endoscope or larger sized medical instrument may be inserted, and an auxiliary tube for monitoring carbon dioxide exhalation by the patient.

### CROSS-REFERENCES TO RELATED APPLICATIONS

Not applicable.

### STATEMENT REGARDING FEDERALLY SPONSORED RESEARCH OR DEVELOPMENT

Not applicable.

### NAMES OF THE PARTIES TO A JOINT RESEARCH AGREEMENT

Not applicable.

### REFERENCE TO A “SEQUENCE LISTING,”A TABLE, OR A COMPUTER PROGRAM LISTING APPENDIX

Not applicable.

### BACKGROUND OF THE INVENTION

(1) Field of the Invention

The present inventive concept relates to an oxygen mask and more particularly, to an improved non-rebreathing oxygen mask for supplying a continuous flow of oxygen to a patient. The non-rebreathing oxygen mask of the present invention is conventional in construction and generally includes a face mask portion for enclosing the mouth and nose of a patient, an oxygen reserve bag for receiving a supply of oxygen from an external source, and an oxygen entry port, disposed intermediate the face mask portion and said oxygen reserve bag, for regulating the flow of oxygen into the oxygen reserve bag, and the flow of oxygen from the oxygen reserve bag into the face mask portion.

Currently available non-rebreathing oxygen masks generally include the above-described face mask portion, oxygen reserve bag and intermediate oxygen entry port, with the oxygen reserve bag attached around, and extending away from, the distal end of the oxygen entry port. The oxygen reserve bag is typically constructed from a thin plastic material, and is configured in the shape of a bottle having a thin neck area, a wider main body, and a shoulder portion, having a gradually increasing width, joining the neck area and the main body of the oxygen reserve bag.

The face mask portion of a non-rebreathing oxygen mask is secured over the nose and mouth area of a patient using a head strap. When the face mask portion is suitably positioned, the main body of the oxygen reserve bag extends outwardly over the chest of the patient. In normal operation, sufficient oxygen should be supplied to the oxygen reserve bag through the oxygen entry port to continuously inflate the oxygen reserve bag to a certain volume.

(2) Description of the Related Art, including information disclosed under 37 CFR 1.97 and 1.98.

WO2008153830 (A1) (2008 Dec. 18) A disposable anesthesia face mask suitably sized and configured to engage the nose portion, mouth portion and chin portion of a patient's face comprises a cup-like shell or receptacle member and a flexible inflatable hollow sealing cushion attached to the shell member on a flange thereon and wherein the shell member includes first passageway means for coupling to a breathing circuit, and a second passageway for connection to a flexible tube which, in turn, is adapted to be connected to a CO2 monitoring machine or capnograph.

US #2006/0231091 A1 (Oct. 19 2006) A non-rebreathing oxygen face mask for hospitals and other clinical settings comprising a face mask, a non-rebreather bag which serves as a reservoir for the aerosol treatment, a one-way valve connected to the bottom of the non-rebreather bag that serves as a nebulizer attachment source, a nebulizer containing medication in the form of a liquid, and plurality of tubes which are connected to the face mask and the nebulizer to supply oxygen and/or air from a gas source is disclosed in the present invention. The face mask adheres to the face of a patient with elastic straps and serves to deliver air, oxygen, and aerosolized medication to the patient and has vents to exhaust expelled air to the atmosphere.

U.S. Pat. No. 6,763,831 B2 (Jul. 20 2004) Disclosed is a mask designed to facilitate ventilation of a patient. The mask has a face piece, an oropharyngeal airway, and an ambu bag. The oropharyngeal airway is incrementally movable to move the patient's tongue to clear the patient's oropharynx anc. to assist: in seal a t e face mask to the patient's face. A method of use is disclosed.

US #200310196664 A1 (Oct. 23 2003) An inhalation breathing apparatus is provided for administering a respirable fluid, The apparatus is a non-rebreathing face mask, conformable to the contours of an individual's face to enclose the nasal and oral cavities, and includes a source lumen for supplying a respirable fluid, a one-way valve to vent respirated fluid, a reservoir bag coupled to the source lumen and having a one-way valve allowing flow out of the bag and into the mask, and a substantially complaint tubular outer ring disposed on the lip of the mask. The outer ring may be fluid filled and provides a tight fluid seal around the nose and mouth, to prevent the entrainment of room air during administration of respirable fluids such as gaseous oxygen. The body of the mask may also include a number of small openings to allow for the flow or room air into the mask in the event of malfunction.

U.S. Pat. No. 5,492,114 (Feb. 20 1996) A non-rebreathing oxygen mask for supplying a continuous flow of oxygen to a patient. The non-rebreathing oxygen mask includes a face mask portion for enclosing the mouth and nose of a patient, an oxygen reserve bag for receiving a supply of oxygen from an external source, and an intermediate oxygen entry port for regulating the flow of oxygen into the oxygen reserve bag from an external source, and between the oxygen reserve bag and the face mask portion. A spring member, having a first end portion secured to a distal end of the oxygen entry port and a second end freely extending through the neck of the oxygen reserve bag into the interior thereof, is utilized to maintain an unobstructed airflow passageway through the neck area of the oxygen reserve bag.

U.S. Pat. No. 4,50,556 (Apr. 8 1986) The invention discloses an adaptor is disclosed for use with an anesthesia mask to allow an endotracheal. tube and a fiberoptic laryngoscope to be passed through the mask while a patient is being ventilated under general anesthesia. The adaptor comprises a T-shaped member, one limb of which is connected to conventional anesthesia equipment and the other limb of which has a soft, flexible cover with a central opening through which the endotracheal tube and fiberoptic laryngoscope are passed. The flexibility of the cover allows endotracheal tubes of different diameter to be accommodated in the opening, and when the mask is not being used for intubation, the opening in the cover may be obturated by a plug. The adaptor may also include a further plug having a throughbore for insertion in the free end of the endotracheal tube to seal around the laryngoscope.

### BRIEF SUMMARY OF THE INVENTIVE CONCEPT

In order to improve on the prior art non-rebreathing masks, the present inventive concept features a closeable, sealable aperture through which a physician may insert an endoscope or other operating equipment while the patient continues to receive oxygen through the mask. The closeable aperture may comprise an assortment of shapes and configurations and closing means. Another feature of the device is an conjoined auxiliary tube which may be connected to appropriate monitoring equipment to track the volume and rate of expulsion of carbon dioxide by the patient.

### BRIEF DESCRIPTION OF THE VIEWS OF THE DRAWINGS

FIG. 1 depicts a typical prior an version of a non-rebreathing anesthetic mask which has been use for at least three decades.

FIG. 2 illustrates the inventive concept, featuring a non-rebreathing mask having a closeable flap aperture and having two conjoined tubes.

FIG. 3 presents an embodiment of a non-rebreathing mask fabricated with an elastomeric, stacked circular aperture.

FIG. 4 shows an expanded view of a hook-and-felt mechanism which may serve as the closeable aperture for the inventive concept.

FIG. 5 is a perspective view of a stacked, elastomeric circular aperture, useable with the inventive concept.

FIG. 6 depicts a profile view of the elastomeric circular aperture.

FIG. 7 illustrates a hatch closing mechanism useable with the inventive concept.

FIG. 8 shows a closeable latch with a nipple and fitted hole, the latch being suitable for use with the inventive concept.

FIG. 9 presents an angled, perspective view of the closeable latch.

### DETAILED DESCRIPTION OF THE INVENTION

The inventive concept is more thoroughly understood by reference to the series of nine drawing figures accompanying this document. In referring to the figures, the various reference numbered callouts are used to refer to like components throughout the drawings.

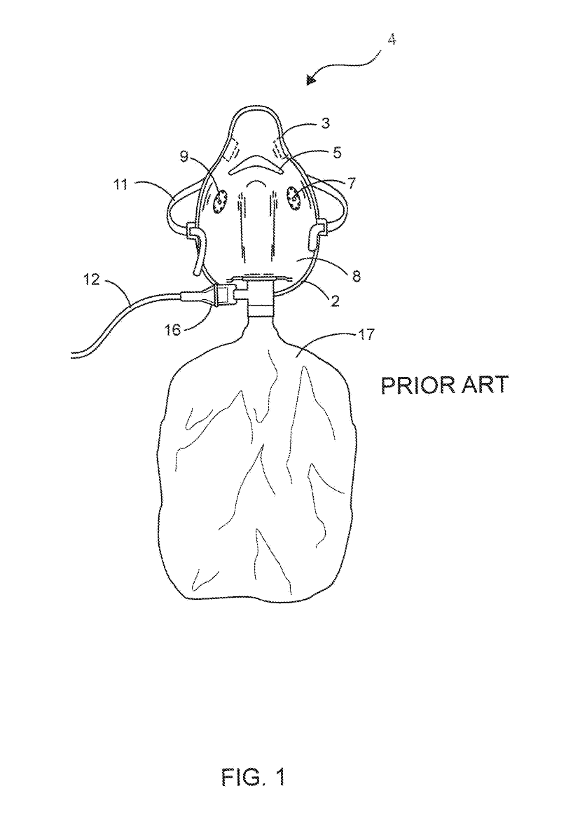

A commonly-used “prior art” non-rebreathing oxygen mask 4 is illustrated in FIG. 1 of this document. The non-rebreathing oxygen mask 4 depicts the mask outer surface 8, the mask peripheral rim 2, a nose bridge section 3 a nose bridge clamp 5 for assisting, in retaining the rim 3 of the mask 4 snug against a patient's face, and left and right exhalation valves 7, 9, which permit breath exhalation by the patient, while cyclically preventing substantial loss of pressurized oxygen flowing to the mask 4.

Also shown in the prior art mask of FIG. 1 is an inflatable, flexible oxygen storage bag 17 for reception of pressurized oxygen, or a mixture of oxygen and air from a supply source. The pressurized oxygen or oxygen mixture is then forced through an oxygen supply tube 12. Once the oxygen storage bag 17 becomes substantially inflated, the pressurized oxygen then continues to flow through the supply tube 12, and an oxygen entry port 16, which port leads to the interior of the non-rebreathing oxygen mask 4. A one-way valve within the oxygen entry port 16 restricts the direction of oxygen flow such that oxygen is only allowed to flow into the oxygen mask 4.

The oxygen reserve bag 17 must be maintained in an inflated condition to at least two-thirds of its full operational capacity. The flow of oxygen through the oxygen supply tube 12 is monitored and controlled, according to the requirements of a specific patient's physical condition and medical background, in order to maintain an appropriate oxygen flow and volume.

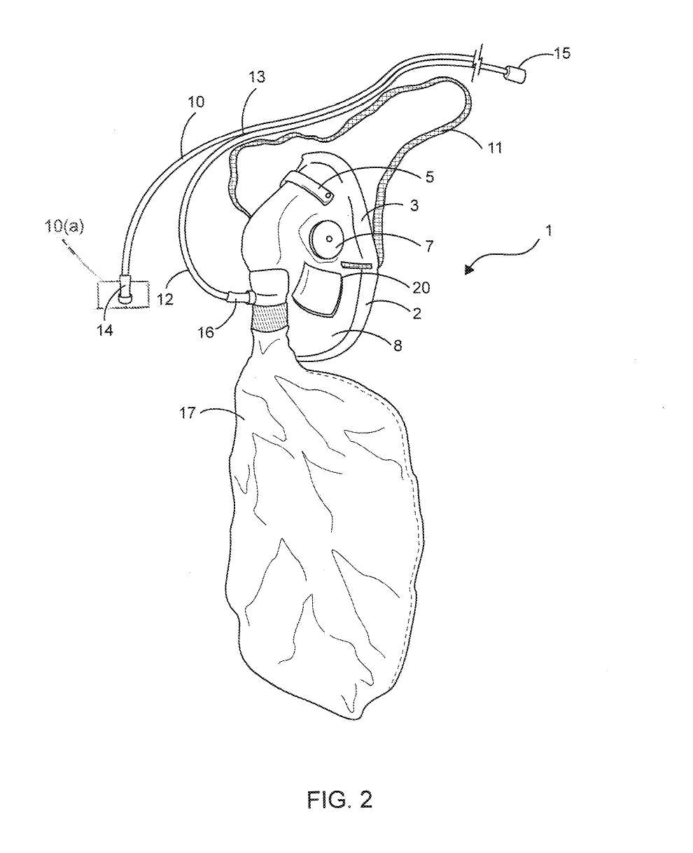

Referring now specifically to FIG. 2, there is shown the present inventive concept, being an improved non-rebreathing oxygen mask 1. This preferred embodiment depicts a closeable bordered flap 20 which may be utilized for insertion of an endoscope or other medical instrument during a surgical procedure. The improved non-rebreathing oxygen mask 1 further comprises a mask outer surface 8, the mask peripheral rim 2, the nose bridge section 3, a nose bridge clamp 5, the left exhalation valves 7, and an inflatable, flexible oxygen storage bag 17.

An oxygen supply tube 12 is connected to a pressurized oxygen source by means of a fitting 15. The oxygen or oxygen mix flows through the supply tube 12, through an oxygen entry port 16, and into the oxygen storage bag 17. Once the oxygen storage bag 17 becomes adequately inflated, the oxygen then flows from the oxygen storage bag 17 for inhalation by the patient. Also shown is a conjoined tube 10 which is utilized for measurement and monitoring of a patient's exhaled carbon dioxide during use of the non-rebreathing oxygen mask 1.

Prior to usage, the oxygen supply tube 12 and the conjoined tube 10 are removably attached to each other along the entire length of each tube 10,12. In FIG. 2, the conjoined tube 10 is shown as having been separated from the oxygen supply tube 12 at a selected junction 13. After such separation, the conjoined tube 10 is routed to appropriate equipment, which may be a typical anesthesia machine. The conjoined tube 10 is connected to the equipment by means of an auxiliary fitting 14. After the connection is established, medical personnel will be given a continuous reading of the levels or exhaled carbon dioxide. The conjoined tube 10 may further have a nasal cannula affixed at some point along the length of the conjoined tube 10.

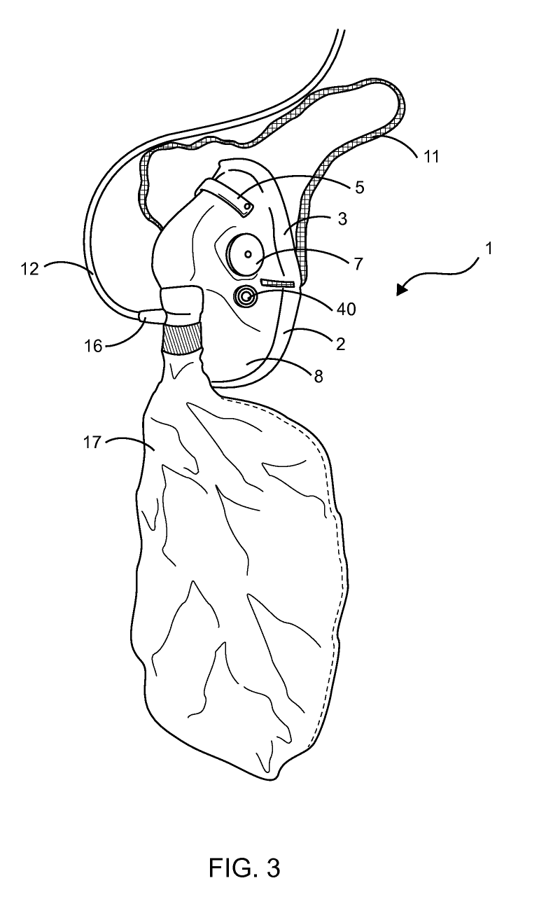

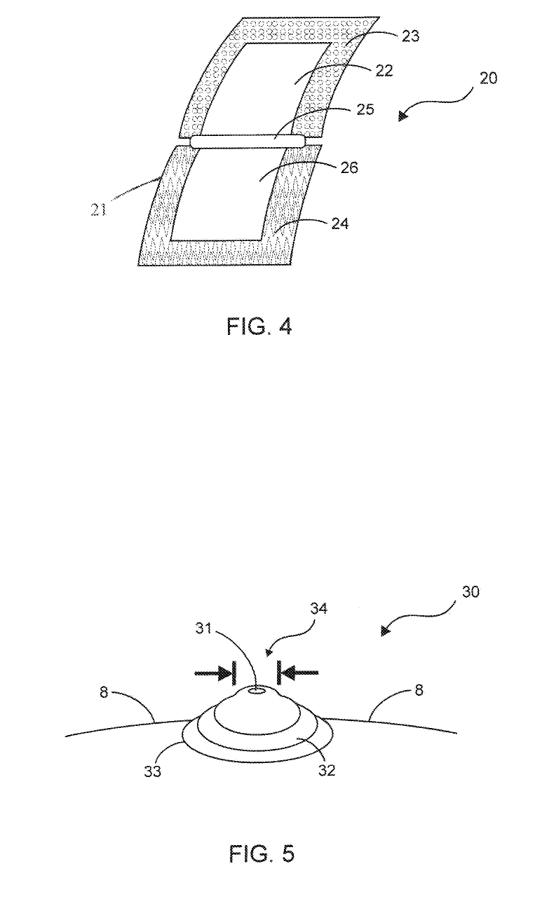

Referring again to the closeable bordered flap 20, this feature is presented in greater detail in FIG. 4 herein. FIG. 4 illustrates the bordered flap 20 being comprised of an upper flap cover 22 having an upper border 23 consisting of the felt component of a hook-and-felt fastening mechanism, and a lower flap base 21 having a lower border 24 consisting of the book component of a hook-and-felt fastening mechanism. Further shown is a hinge 25, which forms a junction between the upper flap cover 22 and the lower flap base 21. The bordered flap 20 further comprises a flap opening 26 through which an endoscope or similarly-sized medical item may be inserted into the interior of the non-rebreathing oxygen mask 1. When the flap opening 26 is not used, the bordered flap 20 may he maintained in the closed configuration by rotating the upper border 23 about the hinge 25 and pressing it onto the lower border 24 to seal the opening.

FIG. 3 depicts a different embodiment of the improved non-rebreathing oxygen mask 1, having an elastomeric aperture 30 comprising an assembly of stacked, conjoined circular ridges. Details of the elastomeric aperture are presented in FIG. 5, where there is shown a lower ridge 33, a middle ridge 32, and an upper ridge 31. An expandable elastomeric opening 34 is defined by a tubular structure penetrating all three ridges 31, 32, 33 of the elastomeric aperture 30.

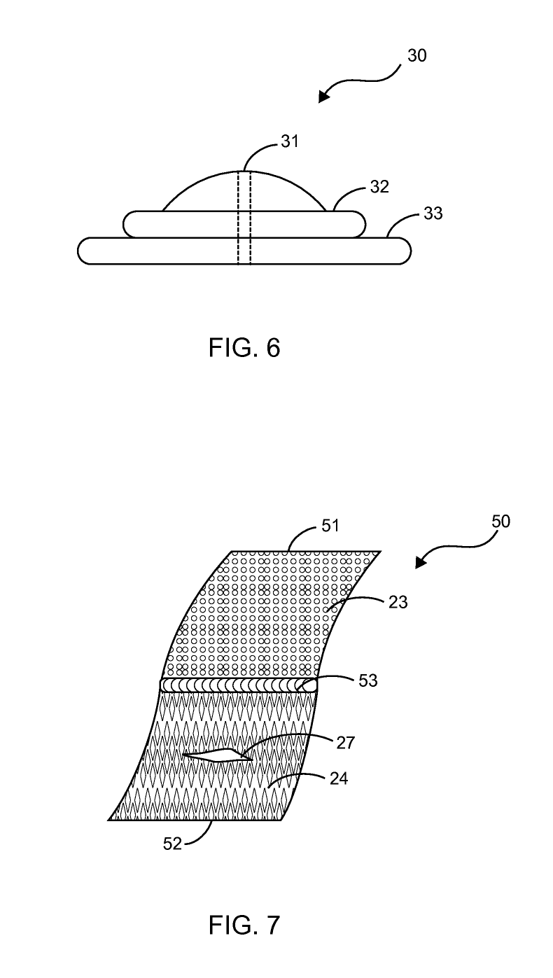

FIG. 6 illustrates, by means of the dashed lines, the tubular interior elastomeric opening 34, which penetrates all three ridges of the elastomeric opening 34. The circumference of the elastomeric opening 34 may comprise a range of width dimensions to accommodate the insertion of an endoscope or other instrument. Further, the elastomeric opening 34 is expandable from an initial circumferential dimension to an opening 25% to 60% larger than the initial undisturbed circumference. The lower ridge 33 is attached, at its undersurface, by either a bonding agent, integral construction, or any other suitable means of attachment, to the outer surface 8 of the mask 1.

In viewing FIG. 7, there is shown a hatch 50 closing mechanism which, in other embodiments, may be used as the closeable aperture in the inventive concept. The hatch 50 essentially comprises a hatch cover 51, a hatch base 52, and a hinge 53. The hinge 53 functions as a revolving junction between the hatch cover 51 and the hatch base 52. The inner surface of the hatch cover 51 further comprises the felt component 23 of a hook-and-felt fastening mechanism and the inner surface of the hatch base 52 comprises the hook component 24 of a hook-and-felt fastening mechanism. The undersurface of the hatch base 52 is attached, by either a bonding agent, integral construction, or any other suitable means of attachment, to the outer surface 8 of the mask 1.

A slit 27 is constructed into the center of the hatch base 52, said slit 27 being of a dimension sufficient to allow accommodation of an endoscope or other similar surgical instrument. When the hatch 50 is not used, or after its utilization for insertion of a medical instrument, the hatch 50 may be maintained in the closed configuration by rotating the hatch cover 51 about the hinge 53 and pressing the hatch cover 51 onto the hatch base 52 to seal the slit 27.

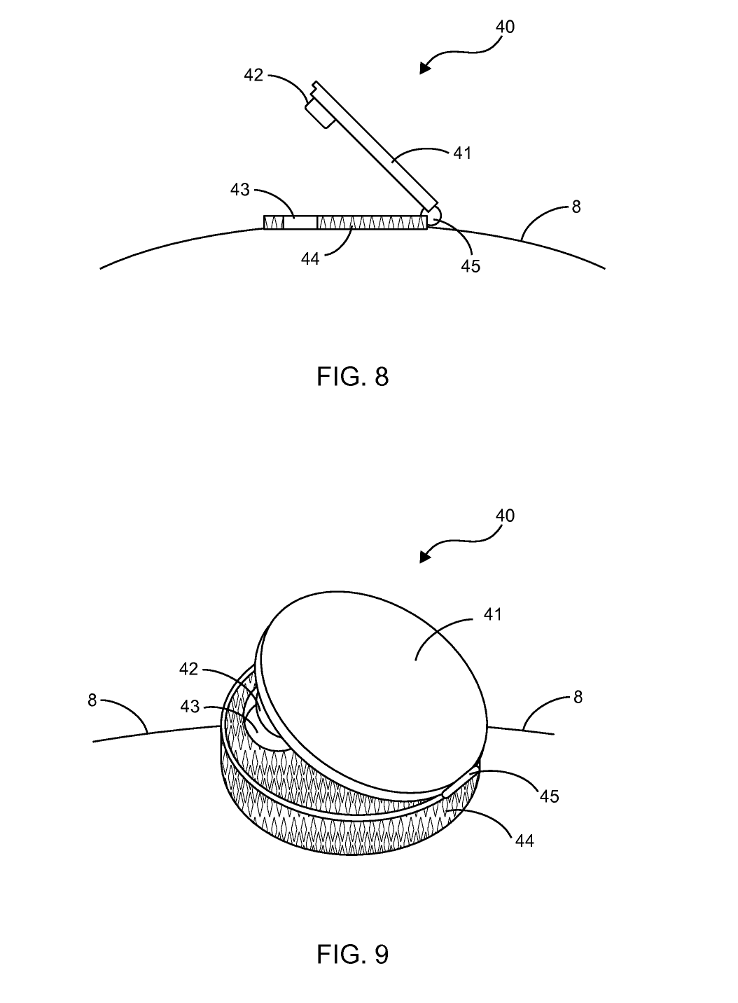

In viewing FIG. 8, there is shown a latch 40 closing mechanism which, in other embodiments, may he used as the closeable aperture in the inventive concept. The latch 40 essentially comprises a latch cover 41, a circular base 44, and a hinge 45. The hinge 45 functions as a revolving junction between the latch cover 41 and the circular base 44. The latch cover 41 further comprises a circular, planar structure, further containing a nipple 42 attached opposite the hinge 45 portion of the latch cover 41. In a similar manner, the circular base 44 comprises a circular, planar structure, further containing a fitted hole 43 reamed into the circular base 44 opposite the hinge 45 portion of the circular base 44, in a location corresponding to the downward positioning of the nipple 42. The circular base 44 is attached, by either a bonding agent, integral construction, or any other suitable means of attachment, to the outer surface 8 of the mask 1.

FIG. 9 depicts a perspective view of the latch 40 as seen with respect to a partially closed device. When the latch 40 is not used, or after its utilization for insertion of a medical instrument, the latch 40 may be maintained in the closed configuration by rotating the latch cover 41 about the hinge 45 and pressing the latch cover 41 such that the nipple 42 engages the fitted hole 43 in the circular base 44.

A typical in-patient medical use of the inventive concept herein is a procedure known as esophagogastroduodenoscopy (EGD). This procedure is a diagnostic endoscopic process that assists the physician in visualizing the upper part of a patient's gastrointestinal tract up to the duodenum. Patients who are anesthesized or have inherent breathing problems may require the administration of oxygen through a non-rebreathing oxygen mask. The inventive concept herein further permits a physician, if necessary, to retroflex the tip or the endoscope into a “J” shape and reinsert through the mask 1 aperture for further examination procedures.

The forewing description of the preferred embodiments of the invention has been presented for purposes of illustration and description. It is not intended to be exhaustive or to limit the invention to the precise form disclosed, and obviously many modifications and variations are possible in light of the above teaching. Such modifications and variations that may be apparent to a person skilled in the art are intended to be included within the scope of this invention as defined by the accompanying claims.

What is claimed is:

1. A non-rebreathing oxygen mask of the type having a face mask tor enclosing the nose and mouth of a person to thereby accommodate a flow of oxygen introduced into said face mask, comprising, outer and inner surfaces, a left exhalation valve, a right exhalation valve, an oxygen supply tube having a connector at its distal end, an inflatable oxygen storage bag having a neck and main body portion, said inflatable oxygen storage bag being constructed from a thin plastic material and said storage bag further including a shoulder portion, said shoulder portion extending between said neck portion and said main body and including an oxygen entry port connecting to the proximal end of said oxygen flow tube, an oxygen flow valve, and a flexible restraining strap, further comprising:
(a) an integral, closeable aperture fabricated proximate the left exhalation valve, said aperture having dimensions which permit the insertion of an endoscope or similarly-sized medical instrument, by exertion of an inward-directed force, from the exterior of said mask, through said aperture and into the interior of said oxygen mask; and
(b) a means for substantially sealing or closing said aperture upon withdrawal of said endoscope or similarly-sized medical instrument from the interior of said oxygen mask, said means selected from the group consisting of a hinged flap mechanism, a hook-and-felt closing mechanism, conjoined, stacked coaxial circular ridges, and a nipple-fitted hole mechanism.

2. A non-rebreathing oxygen mask of the type having a face mask for enclosing the nose and mouth of a person to thereby accommodate a flow of oxygen introduced into said face mask, comprising outer and inner surfaces, a left exhalation valve, a right exhalation valve, an oxygen supply tube having a fitting at its distal end, an inflatable oxygen storage bag having a neck and main body portion, said inflatable oxygen storage bag being constructed from a thin plastic material and said storage bag further including a shoulder portion, said shoulder portion extending between said neck portion and said main body, and including an oxygen entry port connected to the proximal end of said oxygen supply tube, an oxygen flow valve, and a flexible restraining strap, further comprising:
an aperture comprising a hinged flap mechanism having an upper flap cover with an inner perimetral border consisting of the felt component of a hook-and-felt fastening mechanism and a lower flap base having an inner perimetral border consisting of the hook component of a hook-and-felt fastening mechanism, wherein the outer surface of said lower flap base comprises an opening having dimensions which permit the insertion of an endoscope or similarly-sized medical instrument, by exertion of an inward-directed force, from the exterior of said mask, and wherein said lower flap base is permanently attached to the outer surface of said oxygen mask.

3. A non-rebreathing oxygen mask as set forth in claim 2, wherein said hinged flap mechanism comprises an upper flap cover comprising, in its entirety, an inner surface consisting of the felt component of a hook-and-felt fastening mechanism and a lower flap base having a geometric shape corresponding to said upper flap cover and further, said lower flap base having inner surface consisting entirely of the hook component of a hook-and-felt fastening mechanism, and further, wherein said lower flap base is constructed with a slit consisting of dimensions sufficient to allow insertion of an endoscope or other similarly-sized surgical instrument in the center of said lower flap base.

4. A non-rebreathing oxygen mask as set forth in claim 1, wherein said integral, closeable aperture comprises three stacked, conjoined, co-axial circular ridges composed of elastomeric material, further comprising a lower ridge of a first diameter, a middle ridge of a smaller diameter than the lower ridge, and an upper ridge of a smaller diameter than the middle ridge, wherein all three circular ridges are co-axially stacked in order of their decreasing size and wherein a through-opening is formed by a hole reamed through the center of each of said ridges, said through-bole comprising dimensions sufficient to allow insertion of an endoscope or other similarly-sized surgical instrument and further, the lowest of said three circular ridges is permanently attached to the outer surface of said oxygen mask.

5. A non-rebreathing oxygen mask as set forth in claim 1, wherein said integral, closeable aperture comprises a latch closing mechanism which comprises a latch cover of circular, planar structure, a corresponding circular base, and a hinge, said hinge functioning as a revolving junction between the latch cover and the circular base, and further, said latch cover further containing a nipple attached opposite the hinged portion of the latch cover, and further, the circular base contains a fitted hole reamed into the circular base opposite the hinged portion of the circular base in a location corresponding to the downward positioning of said nipple, further, the circular base being attached permanently to the outer surface of said oxygen mask.

6. A non-rebreathing oxygen mask as set forth in any of claim 1, 2, 3, 4, or 5, wherein said oxygen supply tube is conjoined, along the exterior of its length, with an auxiliary tube, said conjoined auxiliary tube having a fitting attached to each its proximal end and its distal end, and a nasal cannula affixed at some point along the length of said conjoined tube.

7. A non-rebreathing oxygen mask as set forth in any of claim 1, 2, 3, 4, or 5, wherein said oxygen supply tube is conjoined, along the exterior of its length, with an auxiliary tube, said conjoined auxiliary tube having a fitting attached to each its proximal end and its distal end.
